(19) United States
(12) Patent Application Publication
(10) Pub. No.: US 2026/0026761 A1
(43) Pub. Date: Jan. 29, 2026

(54) X-RAY IMAGING DEVICE AND X-RAY IMAGING METHOD

(71) Applicant: Hitachi, Ltd., Tokyo (JP)

(72) Inventors: Daiko TAKAMATSU, Tokyo (JP); Akio YONEYAMA, Tokyo (JP); Rika BABA, Tokyo (JP)

(21) Appl. No.: 19/259,080

(22) Filed: Jul. 3, 2025

(30) Foreign Application Priority Data
Jul. 23, 2024 (JP) 2024-117792

(51) Int. Cl. A61B 6/03; A61B 6/00; A61B 6/58; G06T 7/00; G06T 7/11

(52) U.S. Cl. CPC A61B 6/032; A61B 6/5205; A61B 6/586; G06T 7/0012; G06T 7/11; G06T 2207/10081; G06T 2207/20081

(57) ABSTRACT

An X-ray imaging device emits an X-ray to irradiate an object and includes an object moving mechanism that rotates the object; an X-ray image detector to detect a projection image of the object; an object temperature adjusting mechanism configured to change a temperature of the object; and a processing unit configured to acquire a cross-sectional image of the object by a reconstruction calculation from the projection images detected by the X-ray image detector by rotating the object. The processing unit is acquires a plurality of cross-sectional images and of the object captured at a plurality of different temperatures T1 and T2, and divides an inside of the object into regions for each of substances using a distribution of pixels of each of the cross-sectional images based on CT values obtained at the plurality of temperatures T1 and T2 at which the cross-sectional images of the object are acquired.

### CLAIM OF PRIORITY

The present application claims priority from Japanese Patent Application JP 2024-117792 filed on Jul. 23, 2024, the content of which is hereby incorporated by reference into this application.

### BACKGROUND OF THE INVENTION

### 1. Field of the Invention

The invention relates to an X-ray imaging device and an X-ray imaging method for capturing an image of the inside of an object.

### 2. Description of Related Art

An X-ray computed tomography (CT) is a technique that non-destructively obtains cross-sectional images (CT images) of an object by performing a calculation called a reconstruction calculation from a plurality of transmission images (projection images) acquired by rotating the object relative to an X-ray source and projecting X-rays onto the object at different angles. The X-ray CT is an essential technique as a non-destructive visualization technique in a wide range of fields such as medical diagnosis or product inspection because the inside of the object can be three-dimensionally observed non-destructively without cutting the object by utilizing high X-ray transmission.

An example of an X-ray imaging device in the related art is described in PTL 1. In a non-destructive analysis device described in PTL 1, CT imaging is performed at a plurality of timings, CT values at any two points are obtained from the CT imaging, and a temperature of a battery as an object to be inspected is estimated by comparing the CT values.

### CITATION LIST

### Patent Literature

PTL 1: JP2019-90802A

### SUMMARY OF THE INVENTION

In the X-ray CT, a decrease in intensity (linear absorption coefficient) due to absorption of the X-rays generated when the X-rays are transmitted through the object is imaged. The linear absorption coefficient is given by a product of a density and a mass absorption coefficient of the object, and thus two kinds of substances having different densities may also have the same linear absorption coefficient depending on a value of the mass absorption coefficient. In this case, it is difficult to identify the substances having different densities and divide (segment) the inside of the object into regions for each substance.

As a method for solving this problem, a method (dual energy CT) has been developed in which the X-ray CT measurement is performed with a plurality of different X-ray energies, and substances are identified by utilizing the fact that an energy dependence of a linear absorption coefficient is different for each substance. However, in order to implement high identification ability, it is essential to use X-rays with a single energy (monochromatic X-rays). The X-ray CT in the related art uses the polychromatic X-rays (white X-rays) composed of various energies, and there is a problem that X-rays having very high intensity such as radiated light are required in order to perform measurement in a practical time by using the monochromatic X-rays.

In addition to the above, a device (scanning X-ray microscope) has been developed that scans an object with X-rays focused into a pencil shape by an X-ray focusing element and identifies substances by identifying elements based on the energy analysis of fluorescent X-rays generated from irradiation points. However, in such a device, the energy of the fluorescent X-rays is several tens of keV, and the fluorescent X-rays generated inside the object are absorbed by the object, and thus it is difficult to non-destructively measure a deep portion of the object with high definition.

The non-destructive analysis device described in PTL 1 can detect a change in a state inside the object whose structure is known, but it is difficult to divide the inside of an object whose structure is unknown into regions for each of substances.

In this way, the X-ray imaging device in the related art has a problem in dividing the inside of the object into regions for each of substances with high accuracy.

An object of the invention is to provide an X-ray imaging device and an X-ray imaging method capable of dividing the inside of an object into regions for each of substances with high accuracy.

An X-ray imaging device of the invention includes: an X-ray source configured to emit an X-ray to irradiate an object; an object moving mechanism configured to rotate the object; an X-ray image detector configured to detect a projection image of the object made by the X-ray; an object temperature adjusting mechanism configured to change a temperature of the object; and a processing unit configured to acquire a cross-sectional image of the object by a reconstruction calculation from a plurality of the projection images detected by the X-ray image detector by rotating the object. The processing unit is configured to acquire a plurality of the cross-sectional images of the object captured at a plurality of different temperatures, and divide an inside of the object into regions for each of substances using a distribution of pixels of each of the cross-sectional images based on CT values obtained at the plurality of temperatures at which the cross-sectional images of the object are acquired.

An X-ray imaging method of the invention includes: a temperature changing step of changing a temperature of an object by an object temperature adjusting mechanism; an irradiating step of irradiating the object with an X-ray by an X-ray source; a projection image detecting step of detecting, by an X-ray image detector, a projection image of the object made by the X-ray; and a region dividing step of a processing unit, which is configured to acquire a cross-sectional image of the object by a reconstruction calculation from a plurality of the projection images detected by the X-ray image detector by rotating the object, acquiring a plurality of the cross-sectional images of the object captured at a plurality of different temperatures, and dividing an inside of the object into regions for each of substances by using a distribution of pixels of each of the cross-sectional images based on CT values at the plurality of temperatures at which the cross-sectional images of the object are captured.

According to the invention, it is possible to provide the X-ray imaging device and the X-ray imaging method capable of dividing the inside of the object into the regions for each of the substances with high accuracy.

### BRIEF DESCRIPTION OF THE DRAWINGS

FIG. 1A is a diagram illustrating an example of a cross-sectional image of an object at a temperature T1;

FIG. 1B is a diagram illustrating an example of a cross-sectional image of the object at a temperature T2;

FIG. 1C is a diagram illustrating an example of a TT map;

FIG. 1D is a diagram illustrating an example of the TT map in which plotted points are divided into point clouds;

FIG. 1E is a diagram illustrating an example of a cross-sectional image in which regions are divided according to colors, and in which the object is divided into regions;

FIG. 2 is an example of a user interface displayed on a display unit of an X-ray imaging device, which is used when a user divides the points plotted on the TT map into a plurality of point clouds;

FIG. 3 is a diagram illustrating an example of the TT map including point clouds created based on cross-sectional images acquired at temperatures higher and lower than a phase transition temperature;

FIG. 4 is a diagram illustrating an example of data indicating a relation between a volume expansion coefficient and a density of each of typical substances constituting an object;

FIG. 5 is a diagram illustrating an example of a configuration of the X-ray imaging device according to Embodiment 1 of the invention;

FIG. 6 is a flowchart illustrating a procedure of an X-ray imaging method according to Embodiment 1; and

FIG. 7 is a diagram illustrating an example of a configuration of an X-ray imaging device according to Embodiment 2 of the invention.

### DESCRIPTION OF EMBODIMENTS

In the invention, an X-ray CT using polychromatic X-rays (white X-rays) is used to change a temperature of an object, and cross-sectional images of the object are acquired by the X-ray CT at a plurality of different temperatures. Then, a distribution map is calculated based on CT values (linear absorption coefficients) at temperatures for pixels in each of the cross-sectional images, and the inside of the object is divided into regions for each of substances using the distribution map. Hereinafter, this distribution map is referred to as a “temperature-temperature map” (TT map).

According to the invention, it is possible to identify substances or structures inside an object whose internal structure is unknown, and divide the inside of the object into regions for each of the substances or each of the structures with high accuracy.

The basic concept of the invention, that is, a method of dividing the inside of an object into regions for each of substances using cross-sectional images of the object obtained by an X-ray CT, will be described below with reference to the drawings.

In the drawings used in the present specification, the same or corresponding components are denoted by the same reference numerals, and repeated description of these components may be omitted.

In the invention, a temperature T of the object is changed, and cross-sectional images (CT images) of the object are obtained by the X-ray CT at a plurality of different temperatures Tn. A pixel in the CT image has a CT value that represents a linear absorption coefficient of the X-rays.

Hereinafter, an example in which the cross-sectional images of the object are acquired by the X-ray CT at two different temperatures T1 and T2 will be described as an example.

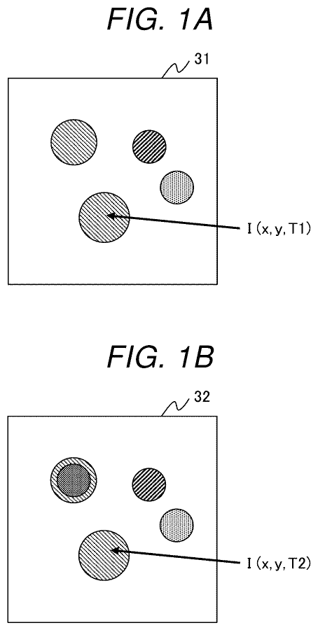

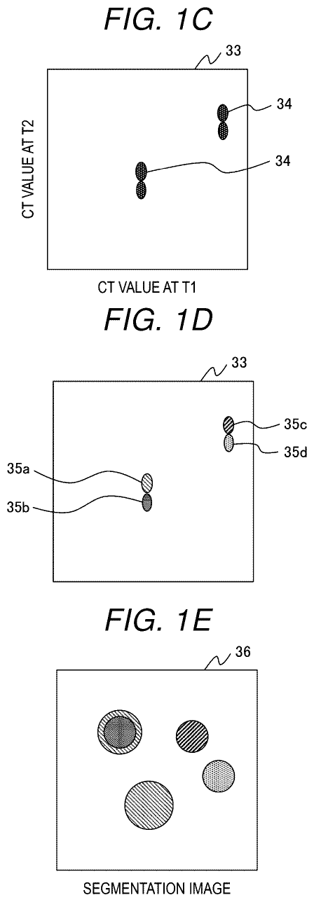

FIG. 1A is a diagram illustrating an example of a cross-sectional image 31 of the object at the temperature T1. FIG. 1B is a diagram illustrating an example of a cross-sectional image 32 of the object at the temperature T2. The cross-sectional image 31 at the temperature T1 represents a CT value I (x, y, T1) in each pixel (x, y) in each of the cross-sectional images (CT images). The cross-sectional image 32 at the temperature T2 represents a CT value I (x, y, T2) in each pixel (x, y) in each of the cross-sectional images (CT images).

In the following description, the object is composed of a plurality of regions divided according to substances constituting the object, and each region is identified by identification number m. These regions can be determined according to the respective densities of the substances constituting the object.

If a density is ρm and a volume expansion coefficient of the region m at the temperature T1 is bm, a density change Δρm when the temperature T of the object changes by ΔT from T1 to T2 is given according to Δρm=bm×ρm*ΔT (1).

On the other hand, if a mass absorption coefficient of the region m is μ′m, a linear absorption coefficient μm is given according to μm=ρm×μ′m (2).

When the mass absorption coefficient μ′m is fixed with respect to the temperature, a change Δμm in the linear absorption coefficient with respect to the temperature change ΔT is Δμm=bm×ρm×μ′m×ΔT (3) according to Equation (1) and Equation (2).

Therefore, the linear absorption coefficient μm, that is, an X-ray CT value of each region changes with a proportional coefficient of bm×ρm×μ′m with respect to the change ΔT in the temperature T.

Therefore, when the X-ray CT values acquired at the different temperatures T are compared with each other, it is possible to accurately divide the object into a plurality of regions determined according to the volume expansion coefficient or the density based on a difference in the density ρm, the volume expansion coefficient bm, and the mass absorption coefficient μ′m. In addition, in the invention, even if no absolute density is detected, the object can be divided into regions determined according to a relative density change of the object accompanying the temperature change.

In the related art, for example, in the technique described in PTL 1, for a CT value I (x, y, Tn) at each pixel (x, y) in each of the cross-sectional images acquired at the plurality of different temperatures Tn, a change in state at each pixel (x, y) is determined by simple subtraction or division. This method can detect the change in the state inside the object whose structure is known, but it is difficult to divide the inside of an object whose structure is unknown into regions for each of substances (densities).

In the invention, the inside of the object is divided into the regions for each of the substances according to the densities thereof, using a procedure to be described below.

Procedure 1) For a pixel (xn, yn) in each of the cross-sectional images acquired at the temperatures T1 and T2, based on a CT value at the pixel (xn, yn), a point is plotted at a position at which a horizontal axis is the CT value at the temperature T1 and a vertical axis is the CT value at the temperature T2. That is, the point plotted corresponding to the pixel (xn, yn) has a horizontal position which is a CT value I (xn, yn, T1) at the temperature T1 and a vertical position which is a CT value I (xn, yn, T2) at the temperature T2.

Procedure 2) The process of Procedure 1) is performed for all pixels of each of the acquired cross-sectional images to create a TT map. As described above, the TT map is a distribution map indicating a distribution of the pixels in each of the cross-sectional images based on the CT values at the plurality of temperatures (a plurality of measured temperatures) at which the cross-sectional images of the object are acquired. Points plotted on the TT map are points corresponding to the pixels of each of the cross-sectional images.

FIG. 1C is a diagram illustrating an example of a TT map 33. In the TT map 33, the pixels in each of the cross-sectional images are plotted as a plurality of points 34 when the horizontal axis is the CT value at the temperature T1 and the vertical axis is the CT value at the temperature T2.

Procedure 3) The points 34 plotted on the TT map 33 are divided into a plurality of point clouds (groups of points that can be regarded as one set), and different numbers (identification numbers) are assigned to the respective point clouds. These numbers are assigned different colors.

The point cloud of the TT map 33 represents a set of pixels having the same physical properties (for example, the same volume expansion coefficient). That is, dividing the points 34 (pixels in each of the cross-sectional images) into the plurality of point clouds means dividing the pixels into the pixels having the same physical properties. A group of pixels having the same physical properties can be obtained from the TT map 33.

FIG. 1D is a diagram illustrating an example of the TT map 33 in which the plotted points 34 are divided into point clouds. In the example illustrated in FIG. 1D, the plotted points 34 are divided into four point clouds 35a to 35d. That is, it can be seen from the TT map 33 illustrated in FIG. 1D that there are regions divided according to four types of physical properties (for example, the volume expansion coefficient).

The point clouds 35a to 35d are assigned numbers (identification numbers) for identifying the respective point clouds 35a to 35d. These identification numbers are assigned different colors. In FIG. 1D, different colors are indicated by different hatching.

Procedure 4) A color assigned to the identification number assigned to each pixel group (that is, each of the point clouds 35a to 35d) is assigned to each pixel (x, y), and a cross-sectional image using the pixel (x, y) to which the color is assigned is newly generated. In the newly generated cross-sectional image, regions are divided according to colors. That is, the region division of the inside of the object is performed based on the pixel groups (point clouds 35a to 35d).

FIG. 1E is a diagram illustrating an example of a cross-sectional image 36 in which regions are divided according to colors, in which the object is divided into regions. In the example illustrated in FIG. 1E, the cross-sectional image 36 (segmentation image) is divided into four regions having different colors. In FIG. 1E, different colors are indicated by different hatching. As illustrated in FIG. 1E, the object can also be divided into regions according to the relative density without obtaining the absolute density.

In the invention, as described above, it is possible to divide the inside of the object into the regions for each of the substances with high accuracy using the cross-sectional images of the object obtained by the X-ray CT.

In the above-described description, an example has been described in which the cross-sectional images of the object are acquired at the two temperatures (T1 and T2). However, the cross-sectional images of the object can be acquired at three or more different temperatures. When the cross-sectional images are acquired at three or more temperatures, a region identification ability can be improved.

In addition, in the above-described description, the TT map 33 is created based on a two-dimensional cross-sectional image obtained by the X-ray CT, and it is also possible to create a TT map based on a three-dimensional cross-sectional image obtained by the X-ray CT and use the TT map to divide the inside of the object into regions in three dimensions.

In Procedure 3, the points 34 plotted on the TT map 33 may be divided into the plurality of point clouds automatically by the X-ray imaging device using any automatic classification algorithm, or manually by the user while viewing the TT map 33 displayed on the display unit of the X-ray imaging device. The automatic classification algorithm is an algorithm for classifying data based on, for example, a self-organizing map method or machine learning.

FIG. 2 is an example of a user interface displayed on the display unit of the X-ray imaging device, which is used when the user divides the points 34 plotted on the TT map 33 into a plurality of point clouds. The user can divide the points 34 plotted on the TT map 33 into a plurality of point clouds by using the user interface as illustrated in FIG. 2.

When the cross-sectional images of the object are acquired at three or more temperatures, it is not possible to display a TT map as a two-dimensional image. Therefore, it is preferred that the points 34 plotted on the TT map are divided into the plurality of point clouds automatically by the X-ray imaging device using the automatic classification algorithm.

In the invention, the larger the temperature difference ΔT (=|T1−T2|) between the temperatures T1 and T2 at which the cross-sectional images of the object are acquired, the larger the difference Δμm between the linear absorption coefficients μm caused by a difference between the volume expansion coefficients bm, making it possible to perform the region division with higher accuracy.

Furthermore, when the cross-sectional images of the object are acquired at the temperatures higher and lower than the phase transition temperature of substances expected to constitute the object (for example, 0° C. if the substance is water or ice), the substances can be identified with higher accuracy. In general, a density of a substance changes or inverts significantly due to a phase transition. Therefore, on the TT map, the point cloud created based on the cross-sectional images acquired at the temperatures higher and lower than the phase transition temperature indicates a peculiar distribution. Therefore, the point cloud can be identified from other point clouds with high accuracy, and the region division can be performed with high accuracy.

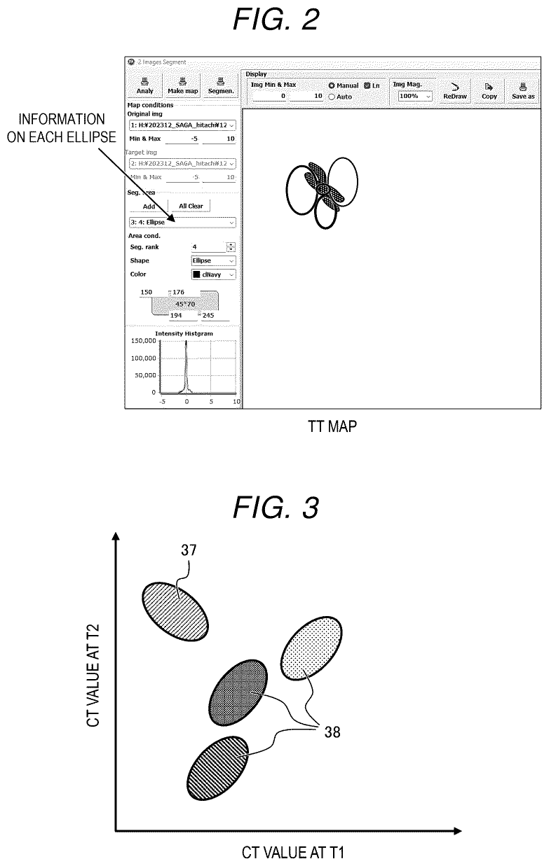

FIG. 3 is a diagram illustrating an example of the TT map including the point clouds created based on the cross-sectional images acquired at the temperatures higher and lower than the phase transition temperature. The TT map illustrated in FIG. 3 indicates a point cloud 37 created based on the cross-sectional images acquired at the two temperatures T1 and T2 higher and lower than the phase transition temperature of a certain substance, and point clouds 38 for substances whose phase transition temperature is not between the temperatures T1 and T2 on the same cross section. In the TT map, a position of the point cloud 37 greatly differs from positions of the point clouds 38. Therefore, when the cross-sectional images of the object are acquired at the temperatures higher and lower than the phase transition temperature, the object can be divided into the regions with high accuracy.

Incidentally, the X-rays are electromagnetic waves with a short wavelength, and when the X-rays are transmitted through the object, in addition to a decrease in intensity due to absorption, a change in phase (phase shift) also occurs at the same time. The X-rays are characterized in that the phase shift is larger by 1,000 times or more than the decrease in intensity with respect to light elements. Therefore, the X-ray CT (phase CT) using a phase contrast X-ray imaging method for imaging a phase shift is used. When the phase CT is used, a living body soft tissue or an organic material mainly composed of light elements such as oxygen and carbon can be observed with high density resolution. For example, the phase CT is used to observe an organ or the like of a small animal with high definition.

A complex refractive index n of the object is expressed as n=1−δ+iβ (4). In Equation (4), a real portion δ and an imaginary portion β are respectively given according to

$$\begin{matrix}“ Math . 1 ” & \\ δ = \frac{{λ}^{2} r e}{2 π} {\sum }_{j} {N}_{j} ( {Z}_{j} + {f}_{j}^{′} ) & ( 5 )\end{matrix}$$

$$\begin{matrix}“ Math . 2 ” & \\ β = \frac{{λ}^{2} r e}{2 π} {\sum }_{j} {N}_{j} {f}_{j}^{″} . & ( 6 )\end{matrix}$$

Here, λ is a wavelength of the X-ray, re is a classical electron radius (2.818×10⁻¹⁵ m), Nj is an atomic density, Zj is the number of electrons in the atom (atomic number), and f′ and f″ are a real portion and an imaginary portion of anomalous dispersion terms of atomic scattering factors.

When the real portion δ and the imaginary portion β are compared with each other, a difference is between (Z+f′) and f″. A ratio between the two portions is 1,000 times or more and δ>>β, and thus the phase CT has high sensitivity. A phase CT value (phase shift) imaged by the phase CT is the real portion δ in Equation (4), and takes different values depending on the temperature.

The linear absorption coefficient μ is related to the imaginary portion β given according to Equation (6) as follows:

$$\begin{matrix}“ Math . 3 ” & \\ β = \frac{λ}{4 π} μ . & ( 7 )\end{matrix}$$

Furthermore, as shown in Equation (2), the linear absorption coefficient μ is given by a product of the density ρ and the mass absorption coefficient μ′ of the object, and the mass absorption coefficient μ′ is a quantity that depends on the elements.

Therefore, it can be seen that it is not possible to precisely calculate the density based on the linear absorption coefficient μ, that is, the CT value unless elemental compositions of the object are known.

On the other hand, in the phase CT, the real portion δ, that is, (Z+f′) is imaged instead of the imaginary portion β. f′ is as very small as about 1/10,000 as compared with Z. Therefore, the phase CT value (phase shift) imaged by the phase CT is substantially Z, that is, a value proportional to an electron density.

Accordingly, in the phase CT, unlike the X-ray CT in which the linear absorption coefficient is imaged, a density of an object whose elemental compositions are unknown can also be accurately obtained.

In the TT map created based on the cross-sectional images acquired using the phase CT, the phase CT value of the pixel is the density, and the cross-sectional image newly generated in Procedure 4 indicates a density distribution. By using this characteristic, based on the volume expansion coefficient and the density obtained based on phase CT values (or a temperature change ΔT of the CT values) acquired at the plurality of different temperatures (for example, the temperature T1 and the temperature T2), and data indicating a relation between a volume expansion coefficient and a density prepared in advance, a substance or composition of each of the regions constituting the object can be made clear.

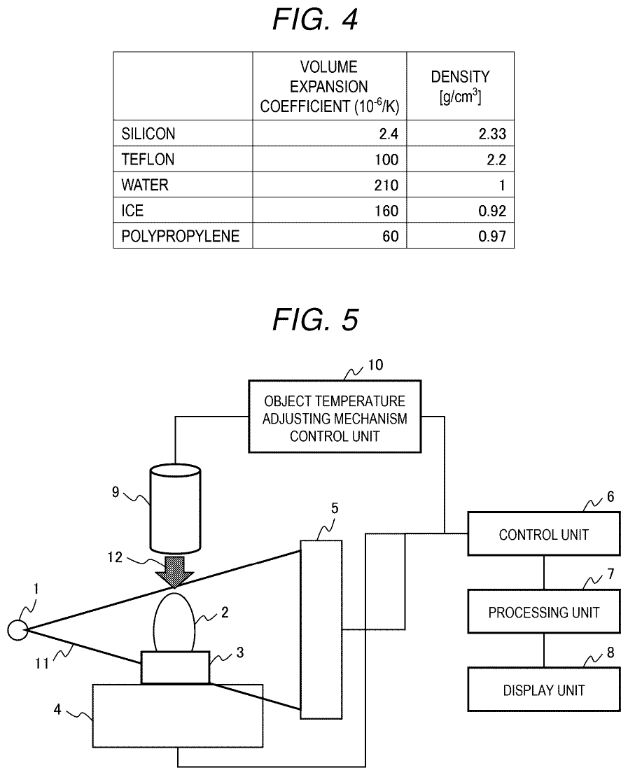

FIG. 4 is a diagram illustrating an example of data indicating a relation between a volume expansion coefficient and a density of each of typical substances constituting the object. The X-ray imaging device according to the invention may include a database in which data as illustrated in FIG. 4 is stored in advance. The X-ray imaging device can not only divide the object into the regions but also specify each of substances constituting each region by collating the phase CT values acquired at the plurality of different temperatures (for example, the temperature T1 and the temperature T2) with the data as illustrated in FIG. 4.

Hereinafter, an X-ray imaging device and an X-ray imaging method according to embodiments of the invention will be described with reference to the drawings.

### Embodiment 1

FIG. 5 is a diagram illustrating an example of a configuration of the X-ray imaging device according to Embodiment 1 of the invention. The X-ray imaging device according to the present embodiment includes an object holder 3, an X-ray source 1, an object moving mechanism 4, an X-ray image detector 5, an object temperature adjusting mechanism 9, an object temperature adjusting mechanism control unit 10, a control unit 6, a processing unit 7, and a display unit 8. The X-ray imaging method according to the present embodiment is executed by the X-ray imaging device according to the present embodiment.

The object holder 3 is provided on the object moving mechanism 4 and holds the object 2.

The X-ray source 1 emits X-rays 11 and irradiates the object 2 held by the object holder 3 with the X-rays 11.

The object moving mechanism 4 is a mechanism that rotates the object 2 by rotating the object holder 3, and adjusts an irradiation position and an irradiation angle of the X-rays 11 on the object 2. In addition, the object moving mechanism 4 can also move the object holder 3 to adjust a position of the object 2 with respect to a position of a beam of the X-rays 11.

The X-ray image detector 5 detects the X-rays 11 transmitted through the object 2 and detects a projection image of the object 2 made by the X-rays 11.

The object temperature adjusting mechanism 9 is a mechanism that changes a temperature of the object 2. FIG. 5 illustrates, as an example, the object temperature adjusting mechanism 9 that changes the temperature of the object 2 by blowing a cooled or heated gas 12 onto the object 2.

The object temperature adjusting mechanism control unit 10 controls the object temperature adjusting mechanism 9.

The control unit 6 controls the object temperature adjusting mechanism 9 to change the temperature of the object 2, and controls the object moving mechanism 4 to rotate and move the object 2, and captures an image of the object 2 by the X-ray CT.

The processing unit 7 acquires cross-sectional images of the object 2 obtained by the X-ray CT from a plurality of projection images detected by the X-ray image detector 5, synthesizes a sinogram image from the cross-sectional images of the object 2, and calculates the cross-sectional images of the object 2 by a reconstruction calculation, thereby acquiring the cross-sectional images of the object 2 obtained by the X-ray CT. Further, the processing unit 7 creates a TT map based on the cross-sectional images of the object 2 acquired at the plurality of temperatures, and divides the object 2 into the regions using the TT map.

The display unit 8 is a display device and displays the cross-sectional images (for example, FIGS. 1A and 1B) of the object 2, the TT maps (for example, FIGS. 1C and 1D), and the cross-sectional image (for example, FIG. 1E) of the object 2 divided into the regions.

The X-ray imaging device according to the present embodiment executes the X-ray imaging method to be described below, and divides the object 2 into the regions.

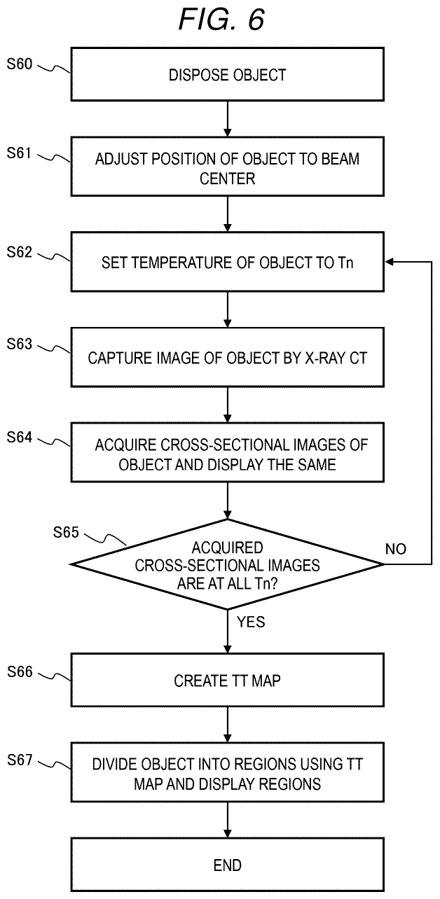

FIG. 6 is a flowchart illustrating a procedure of the X-ray imaging method according to the present embodiment. In the X-ray imaging method according to the present embodiment, cross-sectional images of the object 2 are acquired at a plurality of different temperatures (for example, n temperatures Tn) by the following procedure using the X-ray imaging device according to the present embodiment. The plurality of temperatures (measured temperatures Tn) are predetermined.

In S60, the user disposes the object 2 on the object holder 3.

In S61, the object moving mechanism 4 moves the object holder 3 to align the object 2 with a position of a beam center of the X-rays 11.

In S62, the control unit 6 sends an instruction to the object temperature adjusting mechanism control unit 10 to set the temperature of the object 2 to the measured temperature Tn. The object temperature adjusting mechanism control unit 10 controls the object temperature adjusting mechanism 9 to set the temperature of the object 2 to the measured temperature Tn.

In S63, the X-ray imaging device captures an image of the object 2 by the X-ray CT. First, the object moving mechanism 4 rotates the object holder 3 to rotate the object 2 so that a position at which the X-rays 11 are projected onto the object 2 is a predetermined position (origin). Next, the object moving mechanism 4 moves the object 2 out of an optical path of the X-rays 11, and the X-ray image detector 5 acquires a background image of the object 2. Next, the object moving mechanism 4 returns the object 2 to the optical path of the X-rays 11. Next, the object moving mechanism 4 rotates the object 2 at a preset angle, and the X-ray image detector 5 detects a projection image of the object 2 made by the X-rays 11 at each rotation angle. The rotation of the object 2 and the detection of the projection image are repeated until the rotation angle of the object 2 reaches 180 degrees or 360 degrees. When the rotation angle of the object 2 reaches 180 degrees or 360 degrees, the object moving mechanism 4 moves the object 2 out of the optical path of the X-rays 11, and the X-ray image detector 5 acquires a background image of the object 2. Then, the object moving mechanism 4 returns the object 2 to the optical path of the X-rays 11.

In S64, the processing unit 7 calculates the cross-sectional images of the object 2 by the reconstruction calculation using the acquired group of projection images of the object 2 and the background image, and acquires the cross-sectional images of the object 2 obtained by the X-ray CT. The display unit 8 displays the cross-sectional images of the object 2.

In S65, the processing unit 7 determines whether the cross-sectional images of the object 2 are acquired at all the predetermined n measured temperatures Tn. If the cross-sectional images of the object 2 are not acquired at all the measured temperatures Tn, the process returns to the step S62, and the cross-sectional images of the object 2 are acquired by changing the temperature of the object 2. If the cross-sectional images of the object 2 are acquired at all the n measured temperatures Tn, the cross-sectional images of the object 2 are acquired at the respective measured temperatures Tn, and thus the process proceeds to the step S66.

In S66, the processing unit 7 creates a TT map based on the cross-sectional images of the object 2 acquired at the respective measured temperatures Tn. The TT map (for example, FIGS. 1C and 1D) is generated according to the above-described procedures.

In S67, the processing unit 7 divides the object 2 into the regions using the TT map. The region division (for example, FIG. 1E) of the object 2 is executed according to the above-described procedures. The display unit 8 displays the cross-sectional images of the object 2 divided into the regions.

As the object temperature adjusting mechanism 9, a mechanism for changing the temperature of the object 2 using heat conduction or a mechanism for changing the temperature of the object 2 by blowing a gas such as cooled or heated dry nitrogen to the object 2 can be used. In the mechanism using heat conduction, for example, the object holder 3 is heated by a heater or cooled by a cooling solvent. In the mechanism for blowing the gas to the object 2, a temperature of the gas can be rapidly changed, and thus the temperature of the object 2 can be changed in a short period of time, and the occurrence of frost on a surface of the object 2 due to cooling can be reduced. Therefore, with the mechanism, a highly accurate cross-sectional image that is not affected by frost can be obtained in a short period of time.

Furthermore, the X-ray imaging device according to the present embodiment can include a non-contact type temperature detection mechanism such as thermography, or a contact type temperature detection mechanism such as a thermocouple. The X-ray imaging device according to the present embodiment can confirm whether the temperature of the object 2 reaches the measured temperature Tn by monitoring the temperature of the object 2 with the temperature detection mechanism, can more accurately set the temperature of the object 2, and can perform measurement with higher accuracy.

The X-ray image detector 5 may be a detector that directly detects incident X-rays, such as an X-ray flat panel or a back-illuminated CCD. In the X-ray image detector 5, a pixel size is fixed, but the X-rays can be detected with high efficiency.

The X-ray image detector 5 may be a detector such as an X-ray image intensifier or a lens-coupling type X-ray detector that converts the incident X-rays into electrons or visible light using a phosphor and then detects the converted electrons or visible light using an imaging element. In the X-ray image detector 5, a magnification of a lens system can be changed, and the X-rays can be detected at any magnification. In addition, no X-ray is emitted to the imaging element, and thus the damage to the imaging element by the X-rays can be significantly reduced as compared with the case in which the incident X-rays are directly detected. Furthermore, the X-rays can be detected under optimal conditions by changing a thickness or type of the phosphor in accordance with measurement conditions.

The processing unit 7 can divide the points plotted on the TT map into the plurality of point clouds by using, for example, the automatic classification algorithm based on the self-organizing map method or the machine learning when the object 2 is divided into the regions using the TT map. As already described, the object 2 may be divided into the regions manually by the user using the user interface as illustrated in FIG. 2 while viewing the TT map displayed on the display unit 8 of the X-ray imaging device.

As described above, in the X-ray imaging device and the X-ray imaging method according to the present embodiment, the inside of the object whose internal structure is unknown can be divided into the regions for each of the substances or each of the structures with high accuracy by using the cross-sectional images of the object acquired by the X-ray CT at the plurality of temperatures.

### Embodiment 2

An X-ray imaging device and an X-ray imaging method according to Embodiment 2 of the invention will be described.

In Embodiment 1, the X-ray CT for imaging the change in the intensity of the X-rays generated when the X-rays are transmitted through the object has been described. In the X-ray imaging device and the X-ray imaging method according to Embodiment 1, there is a problem in that the living body soft tissue or the organic material mainly composed of the light elements having small absorption is observed with high definition. In addition, as described above, the linear absorption coefficient μ imaged in Embodiment 1 is given by the product of the density p and the mass absorption coefficient μ′ of the object, and thus there is a problem in that it is difficult to accurately detect the density of the object unless the elemental compositions are known.

Therefore, in the X-ray imaging device and the X-ray imaging method according to the present embodiment, the X-ray phase change (phase shift) generated due to the object is imaged. In a hard X-ray region, for the light elements, a cross-sectional area in which a phase shift occurs is characterized by being larger than a cross-sectional area in which absorption occurs by 1,000 times or more. Therefore, by using the phase shift, the living body soft tissue or the organic material mainly composed of the light elements can be observed with high sensitivity and high definition. Further, the phase shift is substantially proportional to the density, and thus it is possible to accurately detect the density even for an object whose element compositions are unknown.

In the current technique, it is not possible to directly detect the phase shift. Therefore, the phase shift needs to be detected by being converted into detectable X-ray intensity using an X-ray optical element or the like. Examples of the conversion method include (1) an X-ray interference method using an X-ray interferometer, (2) a refractive contrast method of detecting refraction of X-rays by X-ray diffraction, (3) a Talbot interference method using a Talbot interferometer, and (4) a propagation method using Fresnel fringes. Among these, the Talbot interference method is largely characterized by being used for an X-ray source of a quasi-monochromatic and divergent beam, that is, an X-ray source of a laboratory system. Further, the Talbot interference method has a wide dynamic range related to the density, making it possible to measure composite materials that combine metals and organic materials. In the present embodiment, an example using the Talbot interference method will be described as an example.

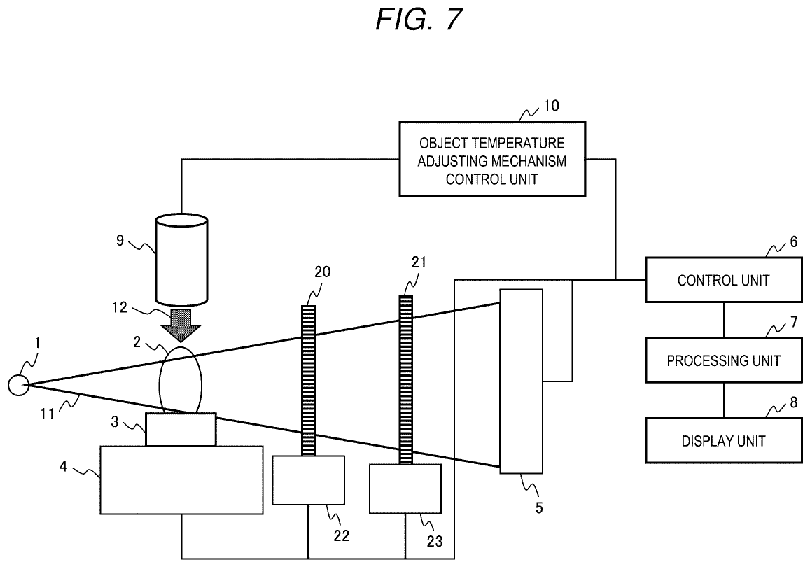

FIG. 7 is a diagram illustrating an example of a configuration of the X-ray imaging device according to Embodiment 2 of the invention. The X-ray imaging device according to the present embodiment is a phase X-ray CT that is provided with a Talbot interferometer, and images an X-ray phase change (phase shift) caused by the object 2 by using the Talbot interference method.

Hereinafter, the X-ray imaging device and the X-ray imaging method according to the present embodiment will be described, focusing mainly on the differences from Embodiment 1.

The Talbot interferometer is a diffraction grating interferometer including a plurality of X-ray diffraction gratings called a phase grating 20 and an absorption grating 21. The X-ray diffraction grating includes a plurality of regions having different thicknesses and different X-ray transmittances in a lattice pattern. Hereinafter, an example in which the Talbot interferometer includes one phase grating 20 and one absorption grating 21 will be described.

The X-ray imaging device according to the present embodiment includes the phase grating 20 and the absorption grating 21 between the object 2 and the X-ray image detector 5, that is, between the object holder 3 and the X-ray image detector 5 in the X-ray imaging device (FIG. 5) according to Embodiment 1. The X-ray imaging device according to the present embodiment further includes a phase grating moving mechanism 22 for relatively moving or rotating the phase grating 20 with respect to the absorption grating 21, and an absorption grating moving mechanism 23 for relatively moving or rotating the absorption grating 21 with respect to the phase grating 20. The phase grating moving mechanism 22 adjusts a position of the phase grating 20 with respect to the object 2. The absorption grating moving mechanism 23 adjusts a position of the absorption grating 21 with respect to the phase grating 20.

The phase grating 20 and the absorption grating 21 are disposed between the object 2 and the X-ray image detector 5. In the phase grating 20 and the absorption grating 21, the phase grating 20 is disposed at a position closer to the object 2 (or the X-ray source 1), and the absorption grating 21 is disposed at a position closer to the X-ray image detector 5. The phase grating 20 is driven by the phase grating moving mechanism 22, and the absorption grating 21 is driven by the absorption grating moving mechanism 23 to determine respective positions with respect to the object 2.

Interference fringes generated by the Talbot interferometer are measured by the X-ray image detector 5.

A phase shift image is quantitatively acquired from a plurality of interference fringe images obtained by relatively moving the phase grating 20 and the absorption grating 21 by 1/n (n is 3 or more) of an interval between the phase grating 20 and the absorption grating 21. The processing unit 7 acquires such interference fringe images from projection images of the object 2 detected by the X-ray image detector 5 (projection images obtained by relatively moving the phase grating 20 and the absorption grating 21). The interference fringe image corresponds to an image obtained by imaging a spatial differential amount of a phase, that is, a spatial differential distribution image of a phase shift.

The processing unit 7 obtains the phase shift by a calculation to be described below. If an interference image acquired at an m-th time is Im, a phase shift φ can be calculated according to

$$\begin{matrix}“ Math . 4 ” & \\ ϕ = {tan}^{- 1} ( \frac{{\sum }_{m = 0}^{n - 1} Im sin \frac{2 π m}{n}}{{\sum }_{m = 0}^{n - 1} Im cos \frac{2 π m}{n}} ) . & ( 8 )\end{matrix}$$

Further, the phase shift q is proportional to a spatial differential amount of the density of the object 2, and thus the spatial distribution image of the phase shift, that is, a density distribution image can be obtained by integrating the obtained phase shift image in the same direction as a direction in which the phase grating 20 and the absorption grating 21 are relatively moved. By performing such a calculation, the processing unit 7 can obtain the density distribution of the object 2 based on the obtained phase shift q.

A procedure of the X-ray imaging method according to the present embodiment is a procedure using the phase X-ray CT using a fringe scanning method instead of using the X-ray CT in the flowchart (FIG. 6) illustrated in Embodiment 1, and the other points are the same as those of Embodiment 1.

In the phase grating 20, it is preferred to use a diffraction grating in which an interval between the gratings (regions with different X-ray transmittance) is several micrometers and the phase of the X-ray is shifted by the ¼ wavelength or the ½ wavelength of the X-ray, that is, a diffraction grating in which a difference in thicknesses between the gratings is ¼ or ½ of the wavelength of the X-ray.

In the absorption grating 21, it is desirable that an interval between the gratings is several micrometers and a thickness of one grating is a thickness for completely absorbing the X-rays. However, even if gold is used, a thickness of several tens of micrometers or more is required, which makes its production difficult, and thus a somewhat thinner diffraction grating, that is, a diffraction grating in which a difference in X-ray transmittance between gratings is approximately 30% or more, may be used for the absorption grating 21. However, in this case, a visibility of the interference image decreases, and thus the density resolution decreases accordingly.

The phase grating 20 and the absorption grating 21 are positioned with respect to the object 2 by the phase grating moving mechanism 22 and the absorption grating moving mechanism 23, respectively. When an axis used for fringe scanning is PZT driving (piezoelectric driving), high-speed scanning can be performed, and data can be acquired in a shorter measurement time.

In the present embodiment, the TT map is calculated in the same manner as in Embodiment 1. However, in the present embodiment, the CT value indicated in the TT map is a phase CT value and is a density distribution image. Therefore, the data indicating the relation between the volume expansion coefficient and the density of each of the substances as illustrated in FIG. 4 is stored in the database in advance, and the data and the density change (volume expansion coefficient) associated with the temperature change obtained based on the TT map are collated with each other, whereby it is possible not only to divide the object into the regions but also to specify each of the substances constituting each region.

For example, by comparing the CT value (phase CT value) at the temperature T1 or the CT value (phase CT value) at the temperature T2 with the volume expansion coefficient which is the data stored in the database, the processing unit 7 can specify each of the substances constituting each region of the object 2 by one or both of the volume expansion coefficient and the density.

As described above, in the X-ray imaging device and the X-ray imaging method according to the present embodiment, by using the Talbot interferometer, it is possible to divide the inside of the object whose internal structure is unknown into the regions for each of the substances or each of the structures with high accuracy, and it is also possible to specify each of the substances constituting each region.

The invention is not limited to the above-described embodiments and may include various modifications. For example, the embodiments have been described in detail to facilitate understanding of the invention, and the invention is not necessarily limited to those including all configurations described above. A part of configurations of one embodiment can be replaced with a configuration of another embodiment. A configuration of another embodiment can be added to a configuration of one embodiment. A part of a configuration in each embodiment may be deleted, or may be added with or replaced with another configuration.

What is claimed is:

1. An X-ray imaging device comprising:
an X-ray source configured to emit an X-ray to irradiate an object;
an object moving mechanism configured to rotate the object;
an X-ray image detector configured to detect a projection image of the object made by the X-ray;
an object temperature adjusting mechanism configured to change a temperature of the object; and
a processing unit configured to acquire a cross-sectional image of the object by a reconstruction calculation from a plurality of the projection images detected by the X-ray image detector by rotating the object, wherein
the processing unit is configured to acquire a plurality of the cross-sectional images of the object captured at a plurality of different temperatures, and divide an inside of the object into regions for each of substances using a distribution of pixels of each of the cross-sectional images based on CT values obtained at the plurality of temperatures at which the cross-sectional images of the object are acquired.

2. The X-ray imaging device according to claim 1, wherein
the processing unit is configured to create a distribution map indicating the distribution of the pixels of each of the cross-sectional images based on a CT value at a first temperature and a CT value at a second temperature among the plurality of temperatures, divide the pixels plotted on the distribution map into a plurality of point clouds, and perform region division based on the point clouds.

3. The X-ray imaging device according to claim 2, wherein
the processing unit is configured to divide the pixels plotted on the distribution map into a plurality of point clouds using an automatic classification algorithm.

4. The X-ray imaging device according to claim 3, wherein
the processing unit uses, as the automatic classification algorithm, an algorithm for classifying data based on a self-organizing map method.

5. The X-ray imaging device according to claim 2, wherein
the first temperature is a temperature higher than a phase transition temperature of the substances constituting the object, and
the second temperature is a temperature lower than the phase transition temperature.

6. The X-ray imaging device according to claim 2, further comprising:
a plurality of X-ray diffraction gratings provided between the object and the X-ray image detector, wherein
the processing unit is configured to obtain a phase shift based on a plurality of interference fringe images obtained from the plurality of diffraction gratings, and obtain a density distribution of the object based on the phase shift.

7. The X-ray imaging device according to claim 6, further comprising:
a database configured to store data indicating a relation between a volume expansion coefficient and a density of each of the substances constituting the object, wherein
the processing unit is configured to specify each of the substances inside the object that is divided into the regions by comparing the CT value at the first temperature or the CT value at the second temperature with the volume expansion coefficient.

8. The X-ray imaging device according to claim 6, wherein
the plurality of diffraction gratings are two diffraction gratings, and
of the two diffraction gratings, the diffraction grating disposed closer to the object has a difference in thicknesses between the gratings of ¼ or ½ of a wavelength of the X-ray, and the diffraction grating disposed closer to the X-ray image detector has a difference in X-ray transmittance between the gratings of 30% or more.

9. The X-ray imaging device according to claim 6, wherein
the plurality of diffraction gratings are two diffraction gratings,
the X-ray imaging device further comprises: a mechanism configured to move one of the diffraction gratings relative to the other diffraction grating, and
the processing unit is configured to acquire the interference fringe images from the projection images of the object obtained by relatively moving the two diffraction gratings.

10. An X-ray imaging method comprising:
a temperature changing step of changing a temperature of an object by an object temperature adjusting mechanism;
an irradiating step of irradiating the object with an X-ray by an X-ray source;
a projection image detecting step of detecting, by an X-ray image detector, a projection image of the object made by the X-ray; and
a region dividing step of a processing unit, which is configured to acquire a cross-sectional image of the object by a reconstruction calculation from a plurality of the projection images detected by the X-ray image detector by rotating the object, acquiring a plurality of the cross-sectional images of the object captured at a plurality of different temperatures, and dividing an inside of the object into regions for each of substances by using a distribution of pixels of each of the cross-sectional images based on CT values at the plurality of temperatures at which the cross-sectional images of the object are captured.

11. The X-ray imaging method according to claim 10, wherein
in the region dividing step, the processing unit creates a distribution map indicating the distribution of the pixels of each of the cross-sectional images based on a CT value at a first temperature and a CT value at a second temperature among the plurality of temperatures, divides the pixels plotted on the distribution map into a plurality of point clouds, and performs region division based on the point clouds.

12. The X-ray imaging method according to claim 11, wherein
in the region dividing step, the processing unit divides the pixels plotted on the distribution map into a plurality of point clouds using an automatic classification algorithm.

13. The X-ray imaging method according to claim 11, wherein
the first temperature is a temperature higher than a phase transition temperature of the substances constituting the object, and
the second temperature is a temperature lower than the phase transition temperature.

14. The X-ray imaging method according to claim 11, wherein
a plurality of X-ray diffraction gratings are provided between the object and the X-ray image detector, and
the X-ray imaging method further comprises:
a density acquisition step of the processing unit obtaining a phase shift based on a plurality of interference fringe images obtained from the plurality of diffraction gratings, and obtaining a density distribution of the object based on the phase shift.

15. The X-ray imaging method according to claim 14, wherein
in the density acquisition step, the processing unit specifies each of the substances inside the object that is divided into the regions by comparing the CT value at the first temperature or the CT value at the second temperature with a volume expansion coefficient of a corresponding one of the substances constituting the object using data indicating a relation between the volume expansion coefficient and a density of the corresponding substance.
